(19) United States
(12) Patent Application Publication
(10) Pub. No.: US 2007/0110715 A1
(43) Pub. Date: May 17, 2007

(54) Treatment of alzheimer's disease

(75) Inventor: Luigi Grimaldi, Enna (IT)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Ares Trading S.A., Aubonne (CH)

(21) Appl. No.: 10/549,031

(22) PCT Filed: Mar. 17, 2004
(86) PCT No.: PCT/EP04/50316

§ 371 Date: Dec. 26, 2006

(30) Foreign Application Priority Data
Mar. 19, 2003 (EP) 03100716.4

(51) Int. Cl. A61K 38/21; A61K 31/55; A61K 31/473; A61K 31/444; A61K 31/445

(52) U.S. Cl. 424/85.6; 514/114; 514/221; 514/319; 514/256; 514/457; 514/411; 514/295; 514/214.03; 514/210.01; 514/297; 514/332

(57) ABSTRACT

The present invention. The invention relates to the use of interferon-β (IFN-β) for treating and/or preventing Alzheimer's disease (AD), Creutzfeld-Jakob disease (CJD) or Gerstmann-Sträussler-Scheinker disease (GSSD). It further relates to the use of IFN-β in combination with an Alzheimer's disease treating agent for treating and/or preventing Alzheimer's disease. The use of IFN-β in combination with a cholinesterase inhibitor for treating and/or preventing early-onset Alzheimer's disease is preferred.

### FIELD OF THE INVENTION

The present invention relates to the treatment of dementias. It relates to the use of interferon β (IFN-β for the manufacture of a medicament for treatment and/or prevention of Alzheimer's disease (AD), Creutzfeld-Jakob disease (CJD) or Gerstmann-Sträussler-Scheinker disease (GSSD). It further relates to the use of IFN-β in combination with an Alzheimer's disease treating agent for the manufacture of a medicament for treatment and/or prevention of AD. It specifically relates to the use of IFN-β in combination with cholinesterase inhibitors (ChEI). Aβ toxicity lowering agents, hormone replacement agents, lipid lowering agents, secretase modulating agents, Aβ aggregation inhibitors, neurofibrilator inhibitors or β-amyloid catabolism inhibitors for the manufacture of a medicament for treatment and/or prevention of AD. In particular, it relates to the use of IFN-β alone or in combination with cholinesterase inhibitors (ChEI), Aβ toxicity lowering agents, hormone replacement agents, lipid lowering agents, secretase modulating agents, Aβ aggregation inhibitors, neurofibrillar inhibitors or β-amyloid catabolism inhibitors for the manufacture of a medicament for treatment and/or prevention of early/onset AD.

### BACKGROUND OF THE INVENTION

Alzheimer's Disease (AD)

Alzheimer's disease (AD) is a progressive neurodegenerative disorder characterized by progressive cognitive impairment (loss of memory, cognition and behavioral stability) due to neuronal loss and resulting in language disorders, problems with judgment, problem solving, planning, abstract thought, apraxia, deficits in visual function and dementia. An age-related increase in prevalence is demonstrated in AD, afflicting approximately 6-10% of the population over age 65 and up to 50% over age 85. AD is the primary cause of dementia and the fourth cause of death after cardiovascular disease, cancer and stroke.

The onset of this disease is characterized by impaired ability to recall recent events, but with disease progression other intellectual skills decline. Later, erratic behavior, delusions, and a loss of control over body functions occur. The diagnosis of Alzheimer's disease is based on well-established criteria (McKhann et al. 1984): definite is reserved for disease confirmed at postmortem examination; probable, for clinical disease without associated illnesses; and possible for those individuals meeting criteria with other illnesses that may cause central nervous system dysfunction such as hypothyroidism or cerebrovascular disease. The clinical diagnosis of disease is based on a combination of the neurological and mental status examination and is reasonably accurate. At death, the most frequent pathological manifestations in brain include specific neuropathological lesions in the limbic and cerebral cortices characterized by intracellular paired helical filaments (PHF) and extracellular amyloid plaques. The primary pathological feature of the disease is the extracellular deposition of fibrillar amyloid and its compaction into senile plaques.

Hence, intra- and extracellular amyloid deposits called neurofibrillary tangles and senile plaques (deposits of fibrillar aggregates), respectively, are associated with Alzheimer's disease. Together with extensive neuronal loss (neurons as well as synapses), they are the hallmark neuropathological features of the disease and are still the only means of confirming diagnosis post-mortem. Neurofibrillary tangles consist primarily of hyperphosphorylated tau (a microtubule assembly protein), while the major fibrillar component of senile plaques is the amyloid-β peptide (Aβ), a 40-42-amino acid fragment of the Alzheimer precursor protein (APP). Analysis of genetic mutations that are responsible for very rarer familial forms of the disease has led to the development of the amyloid cascade hypothesis. It is characterized by the formation and deposition of amyloid fibrils by the normally soluble Aβ peptide, as a result of its overproduction by aberrant proteolytic events and its interactions with pathological chaperones such as Apolipoprotein E and antichymotrypsin. They are minor constituents of senile plaques and have allalic variants that are capable of increasing the proclivity of Aβ to assemble into amyloid fibrils.

The senile plaque is the focus of a complex cellular reaction involving the activation of both microglia and astrocytes adjacent to the amyloid plaque, leading to neuronal damage. In fact, microglia are the most abundant and prominent cellular components associated with these plaques. Plaque-associated microglia exhibit a reactive or activated phenotype. Through the acquisition of a reactive phenotype, these microglia respond to various stimuli, as is evidenced by the increased expression of numerous cell-surface molecules, including major histocompatibility complex (MHC) class II antigens and complement receptors.

Mutations in three genes, the amyloid precursor protein (APP) gene on chromosome 21, the presenilin 1 (PS1) on chromosome 14, and the presenilin 2 (PS2) on chromosome 1, have been found in families with an autosomal dominant Alzheimer's disease with onset as early as the third decade of life. An allelic variant of apolipoprotein-E (APOE) ε4 has also been associated with sporadic and familial disease with onset usually after age 65 years. Mutation in α2-macroglobulin has been suggested to be linked to at least 30% of the AD population. Mutations in the genes causing early-onset disease elevate levels of amyloid B peptide (Aβ1-40 and Aβ1-42). The variant APOE allele may be involved in the removal or degradation of amyloid β. Thus, a common pathway leading to the pathogenesis has been identified by the systematic investigation of families with Alzheimer's disease.

Transmissible Spongiform Encephalopathies (TSEs)

Creutzfeldt-Jakob disease (CJD) and Gerstmann-Straussler-Scheinker disease (GSSD) are transmissible spongiform encephalopathies (TSEs). Spongiform refers to the appearance of infected brains, characterized by holes and resembling like sponges under a microscope. CJD is the most common of the known human TSEs. Other human TSEs include kuru, and fatal familial insomnia (FFI). Kuru was identified in people of an isolated tribe in Papua New Guinea and has now almost disappeared. Fatal familial insomnia and GSSD are extremely rare hereditary diseases, found in just a few families around the world.

Creutzfeldt-Jakob disease (CJD) is an unusual, rare, degenerative, invariably fatal brain disorder, with a prevalence of approximately 1 case per million worldwide, which is about 1/10,000 that of Alzheimer's disease. 85% of cases of CJD are sporadic, with familial and estrogenic (or acquired) cases accounting for the remainder. The onset of symptoms typically arises at about 60, and nearly 90% of patients die within the next year. In sporadic CJD, the disease occurs with no known associated risk factors. In hereditary CJD, there is a familial history of the disease, sometimes with the association of a genetic mutation. Iatrogenic CJD is transmitted by exposure to brain or nervous system tissue, usually through certain medical procedures.

Initially, CJD patents experience problems with muscular coordination; personality changes, including impaired memory, judgment, and thinking; and impaired vision, insomnia, depression, or unusual sensations are other usual symptoms. With disease progression, mental impairment becomes severe. Involuntary muscle jerks called myoclonus can occur as well as blindness. Inability to move and speak might arise and coma is a possible outcome. Pneumonia and other infections often occur in these patients and can lead to death.

There are several known variants of CJD, which differ in the symptoms and course of the disease. The new variant or variant (nv-CJD, v-CJD), begins primarily with psychiatric symptoms, affects younger patients than other types of CJD, and has a longer than usual duration from onset of symptoms to death. In patients with new-variant Creutzfeldt-Jakob disease, symptoms develop at a mean age of 26 years—nearly four decades earlier than in patients with sporadic disease—and many patients present with prominent affective symptoms, including dysphoria, initability, anxiety, apathy, loss of energy, insomnia, and social withdrawal. Another variant, called the panencephalopathic form, occurs primarily in Japan and has a relatively long course, with symptoms often progressing for several years. Some symptoms of CJD can be similar to symptoms of other progressive neurological disorders, such as those mentioned before for AD and others related to Huntington's disease. However, CJD causes unique changes in brain tissue and tends to cause more rapid deterioration of a person's abilities than AD or most other types of dementia.

Gerstmann-Straussler-Scheinker disease is characterized by cerebellar ataxia, progressive dementia, and absent reflexes In the legs and pathologically by amyloid plaques throughout the central nervous system. Onset is usually in the fifth decade and in the early phase ataxia is predominant. Dementia develops later. The course ranges from 2 to 10 years

The diagnosis of CJD is usually not suspected until the neurologic symptoms appear, including cognitive impairment, pain and paresthesias, dysarthria, and galt abnormalities. Myoclonus is a late feature, and startle myoclonus is rarely elicited. Standard diagnostic tests will include a spinal tap to rule out more common causes of dementia and an electroencephalogram (EEG) to record the brain's electrical pattern, which can be particularly valuable because it shows a specific type of abnormality in CJD. Computerized tomography of the brain can help rule out the possibility that the symptoms result from other problems such as stroke or a brain tumor. Magnetic resonance imaging (MRI) brain scans also can reveal characteristic patterns of brain degeneration that can help diagnose CJD. But the only way to confirm a diagnosis of CJD is by brain biopsy or autopsy. Immunodiagnosis of Creutzfeldt-Jakob disease is established with the use of antibodies that recognize both the normal and pathologic isoforms of the prior protein or PrP, with specificity conferred by tissue pretreatment that preferentially degrades the normal protein while sparing the pathologic one.

The leading scientific theory at this time maintains that CJD and the other TSEs are caused not by an organism but by a type of protein called a prion. Prions occur in both a normal form or PrP, which is a harmless protein found in the body's cells; and in an infectious form or PrPSc, which causes disease. The harmless and infectious forms of the prion protein are nearly identical, but the infectious form takes a different folded shape than the normal protein. Sporadic CJD may develop because some of a persons normal prions spontaneously change into the infectious form of the protein and then alter the prions in other calls in a chain reaction. Once they appear, abnormal prion proteins stick together and form fibers and/or dumps called plaques. Fibers and plaques may start to accumulate years before symptoms of CJD begin to appear.

Prion diseases (e.g. CJD and GSSD), like AD, are characterized by extracellular accumulations of amyloid fibrils, consisting of protease-resistant isoforms (PrPSc) of the PrP. Also, like AD, presence of a microglial response in affected areas of the brain has been shown in scrapple and CJD. The multicentric amyloid plaques are composed of protease resistant PrP fragments of 8, 15, and 21-30 kDa. Although the 21-kDa fragment has also been observed in CJD, the 8-kDa fragment appears specific to GSSD. Although there are many neuropathologic similarities, GSSD differs from CJD by the presence of kuru-plaques and numerous multicentric, floccular plaques in the cerebral and cerebellar cortex, basal ganglia, and white matter.

Patients with familial CJD as well as GSSD have mutations in the gene encoding PrP (PRNP). Human prion protein is coded by a single exon on the long arm of chromosome 20. Importantly, at least two mutations in the prion gene (at codons 145 and 183) may cause a disease that clinically mimics AD (see below), and an insertion at base pair 144 may present with a very variable phenotype.

The most common mutation associated with familial CJD is at codon 200 of the prion gene with a slightly earlier average age at onset (55 years) and nearby mutations at codons 208 and 210 found in Italian families. The second most common mutation, at codon 178, produces a disease with an earlier onset (fifth decade) and longer duration (1-2 years). While variant CJD has been linked to transmission of the agent of bovine spongiform encephalopathy, all cases tested to date have been homozygous for methionine at codon 129. Many patients with sporadic Creutzfeldt-Jakob disease have abnormal proteins in their cerebrospinal fluid, particularly the 14-3-3 protein.

In GSSD, the codon 102 mutation is the most frequent (found in several European countries and in Japan). It causes the ataxic form of GSSD: cerebellar syndrome in the third or fourth decade at onset followed by visual, pyramidal and intellectual signs. Death occurs anywhere between 1 and 11 years after onset. Amyloid plaques can be found mainly in the cerebellum. The codon 117 mutation (German and Alsacian families) causes dementia with pyramidal or pseudobulbar signs such as gaze palsies, deafness, pseudobulbar palsy and cortical blindness as well as depressed reflexes and extensor plantars. Amyloid plaques are mono- or multicentric. Other rare mutations include: 198 (one American family), 217 (one Swedish family), 145 (one Japanese patient) and 105 (one case in Japan). Multicentric plaques and neurofibrillar degeneration similar in AD are found with the codon 198 and 217 mutations. Clinical symptoms related to AD develop with the codon 145 mutation, where amyloid plaques are made of truncated PrP. Finally the codon 105 mutation causes spastic paraparesia with late dementia. Amyloid plaques are mainly localised in the frontal lobe.

There is no treatment that can cure or control CJD. Current treatment for CJD is aimed at alleviating symptoms and making the patient as comfortable as possible. Opiate drugs might relieve pain, and the drugs clonazepam and sodium valproate could relieve myoclonus. Treatments for GSSD are also inexistent. Compounds that may inhibit the conversion of PrP to its pathologic isoforms could be useful, including acridine and phenothiazine derivatives quinacrine and chloropromazine. Some forms of PrP may resist conformational conversion into pathologic isoforms. Overexpression of these “dominant negative” prion proteins can prevent or dramatically slow down the development of scrapple in mice, suggesting that interference with the conversion of PrP to its pathologic state represents an eventual therapeutic approach.

ChE Inhibitors

Acetylcholinesterases or acetylcholine acetylhydrolases (AChE, EC 3.1.1.8) and related enzyme butyrylcholinesterase or acylcholine acylhydrolases (BuChE, EC 3.1.1.7) are other proteins that are found to be abnormally associated with senile plaques in Alzheimer's disease (1). Studies have indicated that both enzymes may co-regulate levels of the neurotransmitter acetylcholine (ACh) by hydrolysis at cholinergic synapses and neuromuscular junctions in the mammalian nervous system (2) and could play important roles in the brain of patients with AD. The hydrolysis reaction proceeds by nucleophilic attack to the carbonyl carbon, acylating the enzyme and liberating choline. This is followed by a rapid hydrolysis of the acylated enzyme yielding acetic acid, and the restoration of the enzyme. AChE preferentially hydrolises acetylesters such as ACh whereas BuChE preferably other types of esters such as butrylcholine. Three different AChE subunits exist and arise by alternative mRNA splicing: a synaptic Ach E (AChE-S), a hematopoletic AChE (AChE-H) found on red blood cells and a “read-through” AChE (AChE-R).

Severity of Alzheimer-type neuropathology and more specifically degenerative changes in the basal forebrain reduce the content of AChE and choline acetyltransenase activity (3), which correlates with affected areas (4) and occurs early, being related to the early symptoms. BuChE is normally expressed only at very low levels in the brain (5). There is also a correlation between areas that have high levels of AChE and degenerative areas in Alzheimer's disease (6).

Evidence shows that AChE may have a direct role in neuronal differentiation (7). Transient expression of AChE in the brain during embryogenesis suggests that AChE may function in the regulation of neurite outgrowth (8) and in the development of axon tracts (9). Additionally, the role of AChE in cell adhesion have been studied (10). The results Indicate that AChE promotes neurite outgrowth in neuroblastoma cell line through a cell adhesive role (11). Moreover, studies have shown that the peripheral anionic site of the AChE is involved in the neurotrophic activity of the enzyme (12) and conclude that the adhesion function of AChE is located at the peripheral anionic site (13).

Interaction between AChE (but not BuChE) and fibrillar Aβ has been demonstrated (14), and AChE was shown to behave like a pathological chaperone (capable of increasing the rate of fibril formation by Aβ (15) and the neurotoxicity of the fibris (16). AChE directly promotes the assembly of βA peptide into amyloid fibrils forming stable βA-AChE complexes that are able to change the biochemical and pharmacological properties of the enzyme and cause an increase in the neurotoxicity of the βA fibrils. It has also been shown that the neurotoxicity of Aβ peptide aggregates depends on the amount of AChE bound to the complexes, suggesting also that AChE plays a role in the neurodegeneration in AD brain. BuChE is reported to be associated with amyloid plaques. The presence of a fibrillogenic region within AChE may be relevant to the interaction of AChE with amyloid fibrils formed by Aβ (17) and human recombinant acetylcholinesterase (HuAChE) inhibitors were found to inhibit HuAChE-induced Aβ aggregation (18). Hence, regions related to noncholinergic functions of the AChE, such as adhesion and Aβ deposition have been identified. Enhancement of AChE activity within and around amyloid plaques was shown to be induced by Aβ2-35 mediated by oxidative stress, and that vitamin E and NOS inhibitors prevented this effect further suggesting an important role in the maintenance of acetylcholine synaptic levels, thus preventing or improving cognitive and memory functions of AD patients (19).

Thus, cholinergic deficits (particularly loss of cortical cholinergic neurotransmission) are correlated with cognitive impairment and mental functions associated with AD. The development of the first effective symptomatic therapies for mild to moderate AD (20) involves Cholinesterase inhibitors (ChEI) that act by inhibiting the degradation of Ach (21). The clinical efficacy of these drugs has been characterized by cognitive, functional, and global improvements in patients with AD, and there is evidence that they may delay the progression of dementia (21). Cholinergic drugs might be effective in all forms of AD (mild, moderate and severe). Although neocortical cholinergic deficits are characteristic of severely demented patients in AD, overt cholinergic deficits do not generally appear until relatively late in the course of the disease (22). Hence, ChEI showed efficacy in patients with ‘moderate-to-severe’ AD (23). Furthermore, Galantamine showed to patients with ‘advanced moderate’ AD, raising further the possibility of using ChEI not only in mild-to-moderate AD (23).

Inhibitors of AChE act on two target sites on the enzyme, the active site and the peripheral site. Inhibitors directed to the active site prevent the binding of a substrate molecule, or its hydrolysis, either by occupying the site with a high affinity (tacrine) (24) or by reacting irreversibly with the catalytic serine (organophosphates and carbamates) (25). The peripheral site consists of a less well-defined area, located at the entrance of the catalytic gorge. Inhibitors that bind to that site include small molecules, such as propidium (26) and peptide toxins as fasciculins (27). Bis-quaternary inhibitors as decamethonium (28), simultaneously bind to the active and peripheral sites, thus occupying the entire cataytic gorge.

Individual ChEI differ from each other with respect to their pharmacologic properties, and these differences may be reflected in their efficacy or safety profiles. Tacrine, donepazil, and galantamine are reversible ChEI, metrifonate is an irreversible ChEI, and rivastigmine is a pseudo-irreversible (slowly reversible) ChEI with an intermediate duration of action. Whereas the primary target of these agents is AChE, some also show an affinity for BuChE. Some inhibitors (e.g. galantamine) have also a dual mode of action, modulating nicotinic acetylcholine receptors and inhibiting AChE (23). This pharmacological property has been associated with the ability of nicotine and other related α7-receptor agonists to offer neuroprotection in a variety of experimental models (29). The combination of AChE inhibition and nicotinic acetylcholine receptor modulation is suggested to offer potential significant benefits over AChE inhibition alone in facilitating acetylcholine neurotransmission (30). Choline was shown to have both α7-nicotinic agonist activity and potential neuroprotective ability and many of these compounds, including pyrrolidinecholine, are transported along with choline into the CNS (29). Other compounds show also a dual inhibitory mode against AChE and monoamine oxidase (MAO). Rasagiline, selegiline and tranylcypromine are MAO inhibitors that are likely to delay the further deterioration of cognitive functions to more advanced forms in AD. Imino 1,2,3,4-tetrehydrocylopent[b]indole carbamates (hybrids of the AChE inhibitor physostigmine and MAO inhibitors selegiline and tranylcypromine), N-Pyrimidine 4-acetylaniline derivatives, 7-aryloxycoumarin derivatives, propargylamino carbamates such as N-propargylaminoindans and N-propargylphenhylamines are compounds showing dual MAO-AChE inhibitory activity.

Considering the non-cholinergic aspects of the cholinergic enzyme AChE, their relationship to Alzheimer's hallmarks and the role of the peripheral site of AChE in all these functions as well as dual site inhibitors of AChE and dual mode inhibitors such as AChEI with α7 receptor agonists or with MAO inhibitors, cognitive deficit alleviation and β-amyloid assembly reduction might simultaneously occur delaying efficiently the neurodegenerative process.

Hence, inhibitors of cholinesterase, tacrin, amiridine, donepazil and derivative TAK-147 and CP-118954, minaprine, rivastigmine, galantamine, huparzine, huprine, bis-tetrahydroaminoacridine (bis-ThA) derivatives such as bis(7)-tacrine, imidazoles, 1,2,4-thiadiazolidinone, benazepine derivatives, 4,4′-bipyridine, indenoquinolinylamine, decamethonium, edrophonium, Bw284C51, physostigmine derivative eptastigmine, metrifonate, propidium, fasciculins, organophosphates, carbamates, imino 1,2,3,4-teatrahydrocyclopent[b]indole carbamates (hybrids of the AChE inhibitor physostigmine and MAO inhibitors selegiline and tranylcypro mine), N-Pyrimidine 4-acetylanilne derivatives, 7-aryloxycoumarin derivatives, propargylamino carbamates such as N-propargylaminoindans and N-propargylphenethylamines, vitamin E, NOS inhibitors, precursors such as choline and pyrrolidinecholine, as well as cholinergic receptor agonists (e.g. nicotinic, particularly α7 and muscarinic) could be useful in the treatment of AD:

Other Alzheimer Treatments

Aβ TOXICITY REDUCTION: Anti-inflammatory agents could prove useful in AD treatment (31). Nonsteroidal anti-inflammatory drugs such as ibuprofen, indomethacin and sulindac sulfide decrease the amount of Aβ1-42 (32, 33). Death associated protein kinase (DAPK) inhibitors such as derivatives of 3-amino pyridazine could modulate the neuroinflammatory responses in astrocytes by Aβ activation (34). Cyclooxygenases (COX-1 and -2) inhibitors, antioxidants such as vitamins C and E, as well as modulators of NMDA such as memantine could also reduce the cellular toxicity of Aβ. The MAO inhibitors Rasagiline, selegiline and tranylcypromine as mentioned before are likely to delay the further deterioration of cognitive functions to more advanced forms in AD.

HORMONE REPLACEMENT The use of estrogen by postmenopausal women has been associated with a decreased risk of AD (35). Women using hormone replacement had about a 50% reduction in disease risk. Estrogen has been found to exert antiamyloid effects by regulating the processing of the amyloid precursor protein in the gamma secretase pathway (36).

LIPID LOWERING AGENTS AND CHOLESTEROL MODULATION. Lipid-lowering agents (3-hydroxy-3-methyglutaryl coenzyme A (HMG-CoA) reductase inhibitors) or statins are associated with lower risk of AD. Statins were shown to reduce the intra- and extracellular amount of Aβ peptide (37). These agents include methyl-β-cyclodextrin, 7-dehydrocholesterol reductases (e.g. BM15.766), acyl co-enzyme A:cholesterol acyltransferase (ACAT) inhibitors, P13K inhibitors such as wortmannin, lovastatin, pravastatin, atorvastatin, simvastatin, fluvastatin, cerivastatin, rosuvastatin, compactin, mevilonin, mevastatin, visastatin, velostatin, synvinolin, rivastatin, itavastatin, pitavastatin.

SECRETASES INHIBITORS: Inhibitors of β- and γ-secretase (aspartic proteases) are likely to reduce levels of Aβ1-40 and Aβ1-42, and α-secretase promoting molecules could also be useful in the treatment of AD. Aβ peptides are cleaved from APP by the sequential proteolysis by β- and γ-secretases generating Aβ1-40, Aβ1-42 and Aβ-1-43. α-secretase cleaves also APP generating the fragments sAPPα and C83 which are non-amyloidogenic fragments. C83 is then cleaved by γ-secretase, generating the p3 peptide. Inhibitors of β-site amyloid cleaving enzyme (BACE) and BACE2 (β- secretases), which are required for Aβ production, by the use of e.g. peptide inhibitors could be useful as a therapeutic approach to AD (38). Tripeptide aldehyde 1, SIB -1281, OM99-2 and Stat-Val are all peptide inhibitors. Non-peptidic BACE inhibitors include alkoxy substituted tetralins. γ-secretase inhibitors include both peptidic and small molecules such as difluoroketone-based compounds, SIB-1405, hydroxy substituted peptide urea, alanine-phenylglycine derivatives, caprolactams, benzodiazepines and hexanamides. Non-peptidic inhibitors of γ-secretase include fenchylamine sulfonamide, bicyclic sulfonamide and isocoumarin. Probable amyloid production inhibitors through a γ-secretase mechanism further include sulfonamide, diaryl acetylene, imidazopyridine and polyoxygenerated aromatci structures. α-secretase promoting molecules include protein kinase C activators, glutamate, carbachol, muscarinic agonists, AIT-082 (Neotrophin™), neurotrophic agents, coper (II) containing compounds and cholesterol depleting agents.

Aβ AGGREGATION INHIBITORS: Aβ can aggregate into neurotoxic oligomers and fibrils once cleaved from APP. Peptidyl inhibitors (e.g. pentapeptide inhibitors ) are Aβ fragments or fragments analogs from the central hydrophibic region (Aβ10-25) of the peptide, which bind Aβ and alter the formation of Aβ aggregates. Non peptidyl inhibitors are analogs of the amyloid binding dyes Congo red and thioflavin T, analogs of the anticanceragent doxorubicin (e.g. anthracycline -4′-deoxy-4′-iododoxcorubicin (IDOX)), antibodies such as rifampicin or analogs thereof and clioquinol, benzofurans (e.g. SKF-74652), inhibitors of serum amyloid protein (SAP) such as captopril (e.g. CPHPC), and metal chelation by addition of Cu²⁺, ZN²⁺ or Fe³⁺.

NEUROFIBRILLAR INHIBITION: Glycogan synthase indase (GSK3β) and cyclin-dependent kinase 5 (cdkS), which are proline-directed kinases, associate with microtubules, phosphorylate tau at AD-relevant epitopes, and are involved in apoptotic cascades (39) which can be mediated by calpain. GSK3β inhibitors such as LICI, GSK3β and cdk5 inhibitors such as indirubins and paulones, and calpain inhibitors could decrease tau pathology in AD reducing neurofibrillary pathology. Microtubules-stabilizing drugs such as paclitaxel and related agents enhance cell survival and reduce Aβ-induced apoptosis (40).

β-AMYLOID CATABOLISM: Enzymes that degrade amyloid peptides or endogeneous inhibitors of these enzymes could be targets for the treatment of AD (41). Proteolytic enzymes include zinc metalloproteinases (e.g. neprilysin), endothelin-converting enzyme, insulin-degrading enzymes (e.g. IDE, insulysin) and plasmin. Inhibitors of neprilysin have been identified, that could represent targets for drug intervention (41).

Interferons

Interferons are another class of molecules that could prove useful in the treatment of senile dementia.

Interferons are cytokines, i.e. soluble proteins that transmit messages between cells and play an essential role in the immune system by helping to destroy micro-organisms that cause infection and repairing any resulting damage. Interferons are naturally secreted by infested cells and were first identified in 1957. Their name is derived from the fact that they “interfere” with viral replication and production.

Interferons exhibit both antiviral and antiproliferative activity. On the basis of biochemical and immunological properties, the naturally-occurring human interferons are grouped into three major classes: interferon-alpha (leukocyte), interferon-beta (fibroblast) and interferon-gamma (immune). Alpha-interferon is currently approved in is the United States and other countries for the treatment of hairy cell leukemia, venereal warts, Kaposi's Sarcoma (a cancer commonly afflicting patients suffering from Acquired Immune Deficiency Syndrome (AIDS)), and chronic non-A, non-B hepatitis.

Further, interferons (IFNs) are glycoproteins produced by the body in response to a viral infection. They inhibit the multiplication of viruses in protected cells. Consisting of a lower molecular weight protein, IFNs are remarkably non specific in their action, i.e. IFN induced by one virus is effective against a broad range of other viruses. They are however species-specific, i.e. IFN produced by one species will only stimulate antiviral activity in cells of the same or a closely related species. IFNs were the first group of cytokines to be exploited for their potential anti-tumor and antiviral activities.

The three major IFNs are referred to as IFN-α, IFN-β and IFN-γ. Such main lands of IFNs were initially classified according to their cells of origin (leukocyte, fibroblast or T cell). However, it became clear that several types may be produced by one cell. Hence leukocyte IFN is now called IFN-α, fibroblast IFN is IFN-β and T cell IFN is IFN-γ. There is also a fourth type of IFN, lymphoblastoid IFN, produced in the “Namalwa” cell line (derived from Burkitt's lymphoma), which seems to produce a mixture of both leukocyte and fibroblast IFN.

The interferon unit or international unit for interferon (U or IU, for international unit) has been reported as a measure of IFN activity defined as the amount necessary to protect 50% of the cells against viral damage. The assay that may be used to measure bioactivity is the cytopathic effect inhibition assay as described (42). In this antiviral assays for interferon about 1 unit/ml of interferon is the quantity necessary to produce a cytopathic effect of 50%. The units are determined with respect to the international reference standard for Hu-IFN-beta provided by the National Institutes of Health (43).

Every class of IFN contains several distinct types. IFN-β and IFN-γ are each the product of a single gene.

The proteins classified as IFNs-α are the most diverse group, containing about 15 types. There is a cluster of IFN-α genes on chromosome 9, containing at least 23 members, of which 15 are active and transcribed. Mature IFNs-α are not glycosylated.

IFNs-α and IFN-β are all the same length (165 or 166 amino acids) with similar biological activities. IFNs-γ are 146 amino acids in length, and resemble the α and β classes less closely. Only IFNs-γ can activate macrophages or induce the maturation of killer T cells. In effect, these new types of therapeutic agents can be called biologic response modifiers (BRMs), because they have an effect on the response of the organism to the tumor, affecting recognition via immunomodulation.

In particular, human fibroblast interferon (IFN-β) has antiviral activity and can also stimulate natural killer cells against neoplastic cells. It is a polypeptide of about 20,000 Da induced by viruses and double-stranded RNAs. From the nucleotide sequence of the gene for fibroblast interferon, cloned by recombinant DNA technology, (44) deduced the complete amino acid sequence of the protein. It is 166 amino acid long.

A mutation at base 842 (Cys→Tyr at position 141) that abolished its anti-viral activity has been described (45), and a variant done with a deletion of nucleotides 1119-1121.

An artificial mutation was inserted by replacing base 469 (T) with (A) causing an amino acid switch from Cys→Ser at position 17 (46). The resulting IFN-β was reported to be as active as the ‘native’ IFN-β and stable during long-term storage (−70° C.).

Rebif® (recombinant human interferon-β) is a recant development in interferon therapy for multiple sclerosis (MS) and represents a significant advance in treatment. Rebif® is interferon (IFN)-beta 1a, produced from mammalian cell lines. It was established that interferon beta-1a given subcutaneously three times per week is efficacious in the treatment of Relapsing-Remitting Multiple Sclerosis (RR-MS). Interferon beta-1a can have a positive effect on the long-term course of MS by reducing number and severity of relapses and reducing the burden of the disease and disease activity as measured by MRI (The Lancet, 1998).

It has been shown that IFN-β is a potent promoter of nerve growth factor production by astrocytes, and based on this observation it was suggested that IFN-β might have a potential utility in AD, but no experimental data or any other evidences backed up this statement (47).

Most current therapeutic strategies in AD are directed at lowering Aβ levels and decreasing levels of toxic Aβ aggregates through (1) inhibition of the processing of amyloid precursor protein (APP) to Aβ peptide, (2) inhibition, reversal or clearance of Aβ aggregation, (3) cholesterol reduction and (4) Aβ immunization. The present invention involves the use of an interferon-β, alone for the treatment of AD and spongiform encephalopathies or in combination with the aforementioned available AD strategies to produce a synergetic effect for the treatment of AD.

### SUMMARY OF THE INVENTION

The present invention is based on the finding that the administration of IFN-β alone or in combination with Cholinesterase inhibitors (ChEI) has a beneficial effect on early-onset Alzheimer's disease (AD) and significantly reduces clinical signs of the disease in early-onset Alzheimer patients. Based on common features of Alzheimer's disease and spongiform encephalopathies, IFN-β would also be beneficial for Creutzfeld-Jakob disease (CJD) or Gerstmann-Sträussler-Scheinker disease (GSSD).

Therefore, it is a first object of the present invention to use interferon-β (IFN-β), or an isoform, mutein, fused protein, functional derivative, active fraction or salt thereof, for the manufacture of a medicament for treatment and/or prevention of AD, CJD or GSSD.

It Is a second object of the present invention to use IFN-β, or an isoform, mutein, fused protein, functional derivative, active fraction or salt thereof, in combination with an Alzheimer's disease treating agent for the manufacture of a medicament for treatment and/or prevention of AD.

It Is a third object of the present invention to use IFN-β, or an isoform, mutein, fused protein, functional derivative, active fraction or salt thereof, alone or in combination with cholinesterase inhibitors (ChEI), Aβ toxicity lowering agents, hormone replacement agents, lipid lowering agents, secretase modulating agents, Aβ aggregation inhibitors, neurofibrillar inhibitors or β-amyloid catabolism inhibitors for the manufacture of a medicament for treatment and/or prevention in early-onset AD.

It is a fourth object of the present invention to use IFN-β, or an isoform, mutein, fused protein, functional derivative, active fraction or salt thereof, in combination with cholinesterase inhibitors (ChEI), Aβ toxicity lowering agents, hormone replacement agents, lipid lowering agents, secretase modulating agents, Aβ aggregation inhibitors, neurofibrillar inhibitors or β-amyloid catabolism inhibitors for the manufacture of a medicament for treatment and/or prevention of AD.

It is a fifth object of the present invention to use a substance consisting of two separate compositions manufactured in a packaging unit, one composition containing IFN-β and the other one containing an Alzheimer's disease treating agent selected from the groups consisting of cholinesterase inhibitors, Aβ toxicity lowering agents, hormone replacement agents, lipid lowering agents, secretase modulating agents, Aβ aggregation inhibitors, neurofibrillar inhibitors or β-amyloid catabolism inhibitors, for simultaneous, sequential or separate use, but joint administration for the treatment of Alzheimer's disease

It is a sixth object of the present invention to provide for a pharmaceutical composition comprising IFN-β and an Alzheimer's disease treating agent selected from the groups consisting of cholinesterase inhibitors, Aβ toxicity lowering agents, hormone replacement agents, lipid lowering agents, secretase modulating agents, Aβ aggregation inhibitors, neurofibrillar inhibitors or β-amyloid catabolism inhibitors, in the presence of one or more pharmaceutically acceptable excipients.

### DETAILED DESCRIPTION OF THE INVENTION

In accordance with the present invention, it has been found that interferon-β, when administered alone or in combination with a cholinesterase inhibitor (ChEI), have a pronounced beneficial effect on the clinical severity of early-onset Alzheimer's disease (AD). Furthermore, it was shown that IFN-β ameliorates the condition of early-onset AD patients by synergetically enhancing the therapeutic activity of cholinesterase inhibitors in early-onset AD patients. Relying on the fact that IFN-β is a potentor of Alzheimer's disease treating agents (i.e. ChEIs), IFN-β in combination with other Alzheimer's disease treating agents would be beneficial for AD. Based on common features, IFN-β would also be therapeutically useful for songiform encephalopathies like Creutzfeldt-Jakob disease (CJD) or Gerstmann-Sträussler-Scheinker disease (GSSD).

Therefore, one aspect of the invention relates to the use of interferon-β (IFN-β), or an isoform, mutein, fused protein, functional derivative, active fraction or salt thereof, for the manufacture of a medicament for treatment and/or prevention of AD, CJD or GSSD.

In a second aspect, the invention relates to the use of interferon-β (IFN-γ), or an isoform, mutein, fused protein, functional derivative, active fraction or salt thereof, in combination with an Alzheimer's disease treating agent selected from the group consisting of cholinesterase inhibitors, Aβ toxicity lowering agents, hormone replacement agents, lipid lowering agents, secretase modulating agents, Aβ aggregation inhibitors, neurofibrillar inhibitors or β-amyloid catabolism inhibitors for the manufacture of a medicament for treatment and/or prevention of Alzheimer's disease, for simultaneous, sequential or separate use.

Preferably, the invention relates to a particular sub-category of Alzheimer's disease, this sub-category of AD being referred to as an early-onset sub-category.

The term “early-onset AD” herein encompasses the sub-category of patients, wherein the age of onset of AD is consistently before the age of 60 to 65 years and often before age 55 years.

Still preferably, the cholinesterase inhibitor (ChEI) is an acetylcholinesterase inhibitor and/or butyrylcholinesterase inhibitor, or an isoform, mutein, fused protein, recombinant protein, functional derivative, hybrids, variants, active fraction or salt thereof.

Still most preferably, the ChEI is donepezil, rivastigmine, galantamine, tacrine, amiridine, minaprine, huperzine, huprine, bis-tetrahydroaminoacridine (bis-THA), imidazoles, 1,2,4-thiadiazolidinone, benazepine, 4,4′-bipyridine, indenoquinolinylamine, docamethonium, edrophonium, physostigmine, metrifonate, propidium, fasciculins, organophosphates, carbamates, imino 1,2,3,4-tetrahydrocyclopent[b]indole carbamates, N-Pyrimidine 4-acetylaniline, 7-aryloxycoumarin, propargylamino carbamates, vitamin E, NOS inhibitors, ACh precursors such as choline and pyrrolidinecholine, or cholinergic receptor agonists (e.g. nicotinic, particularly α7, and muscarinic).

Still preferably, the Aβ toxicity lowering agents are ibuprofen, indomethacin, sulindac sulfide, death associated protein kinase (DAPK) inhibitors such as derivatives of 3-amino pyridazine, cyclooxygenases (COX-1 and -2) inhibitors, antioxidants such as vitamin C and E, NMDA modulators such as memantine, or MAO inhibitors such as rasagiline, selegiline and tranylcypromine.

Still preferably, the hormone replacement agent is estrogen.

Still preferably, the lipid lowering agents are 3-hydroxy-3-methyglutaryl coenzyme A (HMG-CoA) reductase inhibitors, statins, lovastatin, pravastatin, atorvastatin, simvastatin, fluvastatin, cerivastatin, rosuvastatin, compactin, mevilonin, mevastatin, visastatin, velostatin, synvinolin, rivastatin, itavastatin, pitavastatin, methyl-β-cyclodextrin, 7-dehydrocholesterol reductases, acyl co-enzyme A:cholesterol acyltransferase (ACAT) inhibitors, or P13K inhibitors such as wortmannin.

Still preferably, the secretase modulating agents are inhibitors of β- and/or γ-secretase inhibitors, or α-secretase promoting molecules.

Still most preferably, the β-secretase inhibitors are BACE end BACE2 inhibitors such as tripeotide aldehyde 1, alkoxy substituted tetralins, the γ-secretase inhibitors are difluoroketone-based compounds, hydroxy substituted peptide urea, alanine-phenylglycine derivatives, caprolactams, benzodiazepines, hexanamides, fenchylamine sulfonamide, bicyclic sulfonamide, isocoumarin, diaryl acetylene, imidazopyridine, polyoxygenerated aromatic structures, and the α-secretase promoting molecules are protein kinase C activators, glutamate, carbachol, muscarinic agonists, neurotrophic agents, or coper (II) containing compounds.

Still preferably, the Aβ aggregation inhibitors are peptidyl inhibitors (e.g. pentapeptide inhibitors), analogs of the amyloid binding dyes Congo red and thioflavin T, analogs of the anticanceragent doxorubicin, antibiotics such as rifampicin or analogs thereof and clioquinol, benzofurans, inhibitors of serum amyloid protein (SAP) such as captopril, or metal chelating agents by addition of Cu²⁺, ZN²⁺ or Fe³⁺.

Still preferably, the neurofibrillar inhibitors are GSK3β inhibitors such as LICI, GSK3β and cdk5 inhibitors such as indirubins and paulones, calpain inhibitors, or paclitaxel and related agents.

Still preferably, the β-amyloid catabolism inhibitors are zinc metalloproteinases (e.g. neprilysin), endothelin-converting enzyme, insulin-degrading enzymes (e.g. IDE, insulysin), plasmin, or neprilysin inhibitors.

In a third aspect, the present invention relates to the use of a substance consisting of two separate compositions manufactured in a packaging unit, one composition containing IFN-β and the other one containing an Alzheimer's disease treating agent selected from the groups consisting of cholinesterase inhibitors, Aβ toxicity lowering agents, hormone replacement agents, lipid lowering agents, secretase modulating agents, Aβ aggregation inhibitors, neurofibrillar inhibitors or β-amyloid catabolism inhibitors, for simultaneous, sequential or separate use, but joint administration for the treatment of Alzheimer's disease.

In a fourth aspect, the present invention provides a pharmaceutical composition comprising IFN-β and an Alzheimer's disease treating agent selected from the groups consisting of cholinesterase inhibitors, Aβ toxicity lowering agents, hormone replacement agents, lipid lowering agents, secretase modulating agents, Aβ aggregation inhibitors, neurofibrillar inhibitors or β-amyloid catabolism inhibitors, in the presence of one or more pharmaceutically acceptable excipients.

In accordance with the present invention, the Alzheimer's disease treating agent and the interferon-β may be used simultaneously, sequentially or separately.

The term “cholinesterase inhibitors” may be e.g. a protein, peptide or small molecular weight compound having an inhibitory activity on cholinesterase activity. Such agent may also contribute to cholinesterase degradation, for example. It may also be an agent slowing, decreasing, falling, declining, lessening or diminishing Cholinesterase activity. An agent having, decreasing or inhibiting cholinesterase activity may further be any agent degrading or abolishing the Cholinesterase activity. Examples for such agents include antibodies directed against cholinesterase.

The term “prevention” within the context of this invention refers not only to a complete prevention of the disease or one or more symptoms of the disease, but also to any partial or substantial prevention, attenuation, reduction, decrease or diminishing of the effect before or at early onset of disease.

The term “treatment” within the context of this invention refers to any beneficial effect on progression of disease, including attenuation, reduction, decrease or diminishing of the pathological development after onset of disease.

The term “interferon-β (IFN-β)”, as used herein, is intended to include human fibroblast interferon, as obtained by isolation from biological fluids or as obtained by DNA recombinant techniques from prokaryotic or eukaryotic host cells. The use of Interferons-β or IFN-β of human origin is also preferred in accordance with the present invention. The term interferon-β or IFN-β, as used herein, is intended to encompass salts, isoforms, muteins, fused proteins, functional derivatives, variants, analogs, and active fragments thereof.

A “cholinesterase inhibitor (ChEI)”, as used herein, shall mean both cholinesterase (ChE) inhibitors from plants, insects, fishes, animals or humans, together with naturally occurring alleles thereof.

In one embodiment, the cholinesterase inhibitors, Aβ toxicity lowering agents, hormone replacement agents, lipid lowering agents, secretase modulating agents, Aβ aggregation inhibitors, neurofibrillar inhibitors or β-amyloid catabolism inhibitors are isoforms, muteins, fused proteins, recombinant proteins, functional derivatives, hybrids, variants, active fractions or salts thereof.

In a preferred embodiment, the agent having cholinesterase inhibitory activity is a cholinesterase inhibitor, or an isoform, mutein, fused protein, recombinant protein, functional derivative (e.g. mono-dual—(e.g. huparzine A-tacrine dimaric derivative) or plural-binding site ChE inhibitors), variant, analog, hybrid (e.g. huprine as well as MAO-AChE inhibitors such as 1,2,3,4-tetrahydrocyclopen[b]indole carbamates), active fragment, or salt thereof.

In accordance with the present invention, a cholinesterase inhibitor may also be a molecule inhibiting cholinesterase receptors. Similarly, a secretase inhibitor may also be a molecule inhibiting secretase receptors.

In the following, the “Alzheimer treating agents”, and in particular cholinesterase inhibitors, Aβ toxicity lowering agents, hormone replacement agents, lipid lowering agents, secretase modulating agents, Aβ aggregation inhibitors, neurofibrillar inhibitors or β-amyloid catabolism inhibitors, and most particularly acetylcholinesterase inhibitors or/and butyrylcholinesterase inhibitors, may also be referred to as “substance(s) of the invention”.

As used herein the term “muteins” refers to analogs of a substance according to the invention, in which one or more of the amino acid residues of a natural substance of the invention are replaced by different amino acid residues, or are deleted, or one or more amino acid residues are added to the natural sequence of substance of the invention, without changing considerably the activity of the resulting products as compared to the wild type substance of the invention. These muteins are prepared by known synthesis and/or by site-directed mutagenesis techniques, or any other known technique suitable therefor.

Any such mutein preferably has a sequence of amino acids sufficiently duplicative of that of a substance of the invention, such as to have substantially similar or even better activity to a substance of the invention. The biological function of interferon-β and cholinesterese inhibitors are well known to the person skilled in the art, and biological standards are established and available for IFN-β, e.g. from the National Institute for Biological Standards and Control (http://immunology.org/links/NIBSC).

Bioassays for the determination of IFN-β have been described. An IFN assay may for example be carried out as described by Rubinstein et al., 1981 . Thus, it can be determined whether any given mutein, derivative, hybrid has substantially a similar, or even a better, activity than IFN-β by means of routine experimentation.

Muteins of a substance of the invention, which can be used in accordance with the present invention, or nucleic acid coding thereof, include a finite set of substantially corresponding sequences as substitution peptides or polynucleotides which can be routinely obtained by one of ordinary skill in the art without undue experimentation, based on the teachings and guidance presented herein.

Hybrids, derivatives, mono- dual- plural-binding site ChE inhibitors, variants and analogs of a substance of the invention can be routinely obtained by one of ordinary skill in the ark, without undue experimentation.

Preferred changes for muteins in accordance with the present invention are what are known as “conservative” substitutions. Conservative amino acid substitutions of polypeptides or proteins of the invention, may include synonymous amino acids within a group which have sufficiently similar physicochemical properties that substitution between members of the group will preserve the biological function of the molecule. It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional confirmation, e.g., cysteine residues. Proteins and muteins produced by such deletions and/or insertions come within the purview of the present invention.

Preferably, the synonymous amino acid groups are those defined in Table I. More preferably, the synonymous amino acid groups are those defined in Table II; and most preferably the synonymous amino acid groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins a substance of the invention, for use in the present invention include any known method steps, such as presented in U.S. Pat. Nos. 4,959,314, 4,588,585 and 4,737,462, to Mark et al; 5,116,943 to Koths et al., 4,965,195 to Namen et al; 4,879,111 to Chong at al; and 5,017,691 to Lee et al; and lysine substituted proteins presented in U.S. Pat. No. 4,904,584 (Shaw et al). Specific muteins of IFN-β have been described, for example by Mark et al., 1984.

The term “fused protein” refers to a polypeptide comprising a substance of the invention, or a mutein thereof, fused to another protein, which e.g., has an extended residence time in body fluids. A substance of the invention may thus be fused to another protein, polypeptide or the like, e.g., an immunoglobulin or a fragment thereof.

“Functional derivatives” as used herein cover derivatives of a substance of the invention, and their muteins and fused proteins, which may be prepared from the functional groups which occur as side chains on the residues or the N- or C-terminal groups, by means known in the art, and are included in the invention as long as they remain pharmaceutically acceptable, i.e. they do not destroy the activity of the protein which is substantially similar to the activity a substance of the invention, and do not confer toxic properties on compositions containing It. These derivatives may, for example, include polyathylene glycol side-chains, which may mask antigenic sites and extend the residence of a substance of the invention in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

As “active fractions” of a substance of the invention, or muteins and fused proteins, the present invention covers any fragment or precursors of the polypeptide chain of the protein molecule alone or together with associated molecules or residues linked thereto, e.g., sugar or phosphate residues, or aggregates of the protein molecule or the sugar residues by themselves, provided said fraction has no significantly reduced activity as compared to the corresponding substance of the invention.

The term “salts” herein refers to both salts of carboxyl groups and to acid addition salts of amino groups of the proteins described above or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of the proteins (IFN-β and Alzheimer's disease treating agent, respectively) relevant to the present invention, i.e., the ability to bind to the corresponding receptor and initiate receptor signaling.

One of the most common dementia is Alzheimer. Therefore, in a preferred embodiment of the invention, the use of IFN-β alone or in combination with a cholinesterase inhibitor is used for treatment and/or prevention of Alzheimer disease (AD).

It has been stated that AChEI are more efficient in an early-onset AD, compared to the common form of AD. Therefore, in a most preferred embodiment of the invention, the use of IFN-β alone or in combination with a cholinesterase inhibitor is used for treatment and/or prevention of early-onset Alzheimer disease.

In accordance with the present invention, the use of recombinant human IFN-β and tacrine, amiridine, donepezil derivative TAK-147 and CP-118′954, minaprine, huperzine, huprine, bis-tetrahydroaminoacridine (bis-THA) derivatives such as bis(7)-tacrine, imidazoles, 1,2,4-thiadiazoldinone, benazepine derivatives, 4,4′-bipyridine, indenoquindinylamine, decamethonium, edrophonium, Bw264 C51, physostigmine derivative eptasligmine, metrifonate, propidium, fasciculins, organophosphates, carbamates, imino 1,2,3,4-tetrahydrocyclopen[b]indole carbamates (hybrids of the AChE inhibitor physostigmine and MAO inhibitors selegline and tranylcypromine), N-Pyrimidine 4-acetylaniline derivatives, 7-aryloxycoumarin derivatives, propargylamino carbamates such as N-propargylaminoindans and N-propargylpheneotylamines, vitamin E, NOS inhibitors, precursors such as choline and pyrrodinecholine, as well as cholinergic receptor agonists (e.g. nicotinic, particularly α7, and muscarinic) are specially preferred.

In accordance with the present invention, the use of recombinant human IFN-β and donepezil, rivastigmine or galantamine are most especially preferred.

In a further preferred embodiment, the fused protein comprises an Ig fusion. The fusion may be direct, or via a short linker peptide which can be as short as 1 to 3 amino acid residues in length or longer, for example, 13 amino acid residues in length. Said linker may be a tripeptide of the sequence E-F-M (Glu-Pho-Met), for example, or a 13-amino acid linker sequence comprising Glu-Phe-Gly-Ala-Gly-Leu-Val-Leu-Gly-Gly-Gln-Phe-Met introduced between the sequence of the substances of the invention and the immunoglobulin sequence. The resulting fusion protein has improved properties, such as an extended residence time in body fluids (half-life), increased specific activity, increased expression level, or the purification of the fusion protein is facilitated.

In a preferred embodiment, IFN-β is fused to the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG₂ or IgG₄, or other Ig classes, like IgM or IgA, for example. Fusion proteins may be monomeric or multimeric, hetero- or homomultimeric.

The present invention relates to the single use of interferon-β or its combination with Alzheimer's disease treating agents. The therapeutic entities could also be linked to each other in order to be able to administer one single molecule, be it monomeric or multimeric, instead of two or three separate molecules. A multimeric fusion protein could comprise a cholinesterase inhibitor fused to an Ig moiety, as well as an IFN-β fused to an Ig moiety. If expressed together, the resulting fusion protein, which may be linked by disulfide bridges, for instance, will comprise both the Alzheimer's disease treating agent and IFN-β. The compounds of the present invention may further be linked by any other cross-linking agent or moiety, such as a polyethylene molecule, for instance.

In a further preferred embodiment, the functional derivative comprises at least one moiety attached to one or more functional groups, which occur as one or more side chains on the amino acid residues. Preferably, the moiety is a polyethylene (PEG) moiety. PEGylaton may be carried out by known methods, such as the ones described in WO99/55377, for example.

Human IFN-β dosages for the treatment of AD, CJD or GSSD are ranging from 80 000 IU/kg and 200 000 IU/kg per day or 6 MIU (million international units) and 12 MIU per person per day or 22 to 44 μg (microgram) per person. In accordance with the present invention, IFN-β may preferably be administered at a dosage of about 1 to 50 μg, more preferably of about 10 to 30 μg or about 10 to 20 μg per person per day. The preferred route of administration is subcutaneous administration, administered e.g. three times a week. A further preferred route of administration is the intramuscular administration, which may e.g. be applied once a week.

Preferably 22 to 44 μg or 6 MIU to 12 MIU of IFN-β is administered three times a week by subcutaneous injection.

IFN-β may be administered subcutaneously, at a dosage of 250 to 300 μg or 8 MIU to 9.6 MIU, every other day.

30 μg or 6 MIU IFN-β may further be administered intramuscularly once a week.

IFN-β may also be administered daily or every other day, of less frequent. Preferably, IFN-β is administered one, twice or three times per week.

The administration of active ingredients in accordance with the present invention may be by intravenous, intramuscular or subcutaneous route. The preferred route of administration for IFN-β is the subcutaneous route.

In the treatment of AD, standard dosages of tacrine presently used are 10 mg four times a day, 40 mg/d being the recommended maximum. Presently, capsules of tacrine are taken orally. For donepezil, the standard dosage is 5 mg/d, with a recommended maximum of 10 mg/day. Presently, tablets of donepezil are taken orally. For rivastigmine, 1.5 mg twice a day is the standard dosage, with a recommended maximum of 6 mg twice a day. Presently, capsules of rivastigmine are taken orally. For galantamine, the standard dosage presently used is 4 mg twice a day. Presently, tablets of galantamine are taken orally.

In a preferred embodiment, tacrine is administered at a dosage of about 0.1 to 200 mg per person per day, preferably of about 10 to 150 mg par person per day, more preferably about 20 to 60 mg per person per day, or about 60 to 100 mg per parson per day.

In another preferred embodiment, donepezil is administered at a dosage of about 0.1 to 200 mg per person a day, preferably of about 1 to 100 mg per person a day, more preferably about 2 to 30 mg per person a day, or about 30 to 60 mg per person a day.

In another preferred embodiment, rivastigmine is administered at a dosage of about 0.1 to 200 mg per person a day, preferably of about 0.3 to 50 mg per person a day, more preferably about 0.5 to 20 mg per person a day, or about 20 to 40 mg per person a day.

In another preferred embodiment, galantamine is administered at a dosage of about 0.1 to 200 mg per person a day, preferably of about 0.5 to 100 mg per person a day, more preferably about 1 to 30 mg per person a day, or about 30 to 60 mg per person a day.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

In a preferred embodiment, cholinesterase inhibitors are preferably administered orally.

Depending on the mode of administration, the compounds of the invention can be formulated with the appropriate diluents and carriers to form ointments, creams, foams, and solutions having from about 0.01% to about 15% by weight, preferably from about 1% to about 10% by weight of the compounds.

The term “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, epidural, topical, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

The subcutaneous mute is preferred for IFN-β in accordance with the present invention.

Another possibility of carrying out the present invention is to activate endogenously the genes for the compounds of the invention, i.e. an Alzheimer's disease treating agent and/or IFN-β. In this case, a vector for inducing and/or enhancing the endogenous production of IFN-β and decreasing or inhibiting the endogeneous production of e.g. cholinesterase in a cell normally silent for expression of cholinesterase inhibitors and/or IFN-β, or which expresses amounts of cholinesterose inhibitors and/or IFN-β which are not sufficient, is used for treatment of AD, CJD or GSSD. The vector may comprise regulatory sequences functional in the cells desired to express IFN-β and repress cholinesterase. Such regulatory sequences in the case of IFN-β may be promoters or enhancers, for example and repressors or silencers in the case of cholinesterase. The regulatory sequence may then be introduced into the right locus of the genome by homologous recombination, thus operably linking the regulatory sequence with the gene, the expression of which is required to be induced or enhanced. The technology is usually referred to as “endogenous gene activation” (E.G.A.), and it is described e.g. in WO 91109955.

The invention further relates to the use of a cell that has been genetically modified to produce IFN-β and/or Alzheimer's disease treating agents in the manufacture of a medicament for the treatment and/or prevention of AD and infectious diseases.

For parenteral (e.g. intravenous, subcutaneous, intramuscular) administration, the active protein(s) can be formulated as a solution, suspension, emulsion or lyophilised powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The bioavailability of the active protein(s) according to the invention can also be ameliorated by using conjugation procedures which increase the half-life of the molecule in the human body, for example linking the molecule to polyethylenglycol, as described in the PCT Patent Application WO 92/13095.

The dosage administered, as single or multiple doses, to an individual will vary depending upon a variety of factors, including pharmacokinetc properties, the route of administration, patient conditions and characteristics (sex, age, body weight, health, size), extent of symptoms, concurrent treatments, frequency of treatment and the effect desired.

The substances of the invention may be administered daily or every other day, of less frequent. Preferably, one or more of the substances of the invention are administered one, twice or three times per week.

The daily doses are usually given in divided doses or in sustained release form effective to obtain the desired results. Second or subsequent administrations can be performed at a dosage which is the same, less than or greater than the initial or previous dose administered to the individual. A second or subsequent administration can be administered during or prior to onset of the disease.

According to the invention, the substances of the invention can be administered prophylactically or therapeutically to an individual prior to, simultaneously or sequentially with other therapeutic regimens or agents (e.g. multiple drug regimens), in a therapeutically effective amount. Active agents that are administered simultaneously with other therapeutic agents can be administered in the same or different compositions.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

References to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning an range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now described the invention, it will be more readily understood by reference to the following examples that are provided by way of illustration and are not intended to be limiting of the present invention.

### EXAMPLES

### Example 1

Effect of IFN-β in Combination with an AChEI, in Early-Onset AD Patients

The effect of IFN-β in combination with an AChEI on AD disease development is performed on 40 early-onset AD patients.

The clinical efficacy of IFN-β-1a (Rebif® 22 μg, tiw) in the treatment of AD is evaluated by measuring changes in neuropsyhological performance from baseline.

This 6-month, single-center, pivotal study is performed on 40 early-onset AD patients. Subjects are randomized into two groups: the first group (n=20) receiving Rebif® 22 μg tiw plus an acetylcholinesterase inhibitor (e.g., donepezil, rivastigmine, galantamine, etc.); the second group (n=20) receiving a placebo plus an acetylcholinesterase inhibitor.

Inclusion Criteria

Age≧50 years
Diagnosis of Alzheimer's disease, according to the Diagnostic and Statistical Manual of Mental Disorders, 4th edition (DSM-IV)
Mini-Mental State Examination (MMSE) score of 11 to 25 (inclusive)
Supervision by a caregiver
Given informed written consent and approbation of the Local Ethical Committee
   Exclusion Criteria
Modified Hachinski Ischemic Score ≧4
Unable to undergo neuropsychological evaluation
Significant liver, thyroid or haematological dysfunctions
   Design

Forty patients are randomly assigned, in a double-blind fashion, to receive either Rebif® 22 μg tiw plus an actylcholinesterase inhibitor, subcutaneously, or placebo thw plus an acetylcholinesterase inhibitor, subcutaneously, for 24 weeks.

Sample Size Rationale and Statistical Analyses

The trial is designed as a pilot investigation of the clinical utility of Rebif® 22 μg tiw in combination with an acetylcholinesterase inhibitor in the treatment of AD; sample size was chosen based on feasibility for a single-site study. Continuous variables, including cognitive and behavioral scores, am analysed by measuring changes from baseline; analysis of variance is used to compare between-group differences. Side effects are analysed using descriptive statistics and non-parametric tests.

Assignment

The randomisation schedule is generated in the research pharmacy; the investigator and study personnel remain blinded to the group assignment of participants until the completion of data collections.

Outcome Measures

Outcome measures are assessed at baseline, week 12, and week 25 (study completion).

Primary outcome measures include:

Alzheimer's Disease Assessment Scale (ADAS), cognitive subscale
Global Deterioration Scale
Clinical Global Impression of Change Scale
   Secondary outcome measures include:

MMSE

ADAS, non cognitive subscale

Instrumental Activities of Daily Living (IADL)

Physical Self-Maintenance Scale (PSMS)

Caregiver-rated Global Impression of Change (cGIC)

Evaluation of Adverse Events

The appearance of treatment-related adverse events is assessed at each visit. Withdrawal from the study is warranted upon any of the following:

1) Patient request

2) Investigator request

3) Evidence of severe systemic disease

4) Evidence of severe treatment-related (IFN β-1a) adverse events

### Example 2

Effect of IFN-β in Early-Onset AD Patients

The effect of IFN-β on AD disease development is performed on 40 early-onset AD patients.

The clinical efficacy of IFN-β-1a (Rebif® 22 μg, tiw) in the treatment of AD is determined by measuring differences in neuropsychological performance changes into two treatment arms (placebo and treatment) from baseline to 28-week treatment follow-up.

This 52-week, single-center, pivotal study is performed on 40 early-onset AD patients. Subjects are randomized into two groups: the first group (n=20) receiving Rebif® 22 μg tiw; the second group (n=20) receiving a placebo. The treatment period is ended after 28 weeks.

The investigator and study personnel remain blinded to the group assignment of participants until the completion of data collection.

Inclusion Criteria

Age between 50 and 70 years
Diagnosis of Alzheimer's disease, according to the Diagnostic and Statistical Manual of Mental Disorders, 4th edition (DSM-IV)
Mini-Mental State Examination (MMSE) score of 15 to 25 (inclusive)
Supervision by a caregiver
Given informed written consent and approbation of the Local Ethical Committee
   Study Medication

Rebif® (interferon beta-1a) is supplied in pre-filled syringes containing 0.5 mL. Each syringe contains 22 μg (6 MIU) of interferon beta-1a, 2 mg albumin (human) USP, 27.3 mg mannitol USP, water for injection, and for pH adjustment, acetic acid and/or sodium hydroxide. Rebif is supplied as a sterile solution 22 μg (6 MIU) on 0.5 mL packaged in prefilled syringes intended for SC administration. Rebiject® Mini can be used with the pro-filled syringes of Rebif® solution.

Dose, Route and Schedule of Rebif® Drug Administration

The dosage of Rebif, following initial dose titration, is 22 μg injected subcutaneously three times per week. Rebif is administered, if possible, at the same time (preferably in the late afternoon or evening) on the same three days (e.g. Monday, Wednesday and Friday).

Potential side effects at the onset of treatment may be minimized by a progressive increase in the dose for the first 4 weeks, using the schedule outlined in the table below.

DOSE TITRATION SCHEDULE

| | RECOMMENDED | | |
|---|---|---|---|
| Week | TITRATION | Volume | rebif ® dose |
| 1-2 | 20% | 0.20 mL | 4.4 μg |
| 2-4 | 50% | 0.50 mL | 11 μg |
| >4 | 100% | 1 mL | 22 μg |

Study Decision

Forty patients are randomly assigned in a double-blind, controlled, parallel groups study comparing interferon beta treatment to placebo in patients with Alzheimer's dementia.

Null Hypothesis

Based on the primary objectives of the study (calculated using MMSE and ADAS-cog scores to assess cognitive decline), the null hypothesis is that interferon beta will not stop the progressive decline in cognitive function typical of the natural history of Alzheimer's dementia. In other words, after 12 months of treatment, the MMSE and ADAS-cog scores of patients randomized to receive interferon beta therapy will be similar to those of patients who receive placebo treatment.

Sample Size

For this protocol, patients with an MMSE score equal to 20±5 were enrolled. Sample analyses assumed a clinically relevant effect size coinciding with a standard deviation (SD) respective to mean MMSE and ADAS-cog scores in cohorts of patients enrolled in previous randomized clinical trials. MMSE is a scale with a range from 0 to 30 decreasing with cognitive impairment, abnormal under the value of 26/30 age and education adjusted. ADAS-cog is a test with a score from 0 to 70 that increase with the impairment of cognitive functions, abnormal up a value of 9.5/70. The SDs of mean MMSE and ADAS-cog at baseline have been shown to be equal to approximately 5 and 10, respectively (Farlow R M, Hake A, Messina J, Hartman R, Veach J, Anand R. Response of patients Alzheimer disease to rivastigmine treatment is predicted by the rate of disease progression. Arch Neurol 2001;58:417-22).

On the basis of the enrollment criteria (i.e., patients with mean MMSE scoras equal to 20 and the hypothesis that patients treated with placebo will experience worsening scores of 1.2 points every 3 months (Rogers S L, Friedhoff L T and the Donepezil Study Group. The efficacy and safety of Donepezil in patients with Alzheimer's disease: results of multicentre, randomised, double-bind, placebo-controlled trial. Dementia 1996;7:293-303), the expected mean MMSE score in placebo patients is 15.2. In the case that the null hypothesis is false, the expected mean score in patients treated with interferon beta should be equal to 20.2 (given an SD=5). With respect to the objective of the study, the randomization of 17 patients to each group will permit rejection of the null hypothesis with an alpha equal to 0.05 and power of 80%.

With regards to the primary objective of the effect of interferon beta on cognitive decline evaluated using ADAS-cog, it has been reported in the literature that MMSE scores correspond with ADAS-cog scores (Doraiswamy P M, Bleper F, Kalser L, Krishnan K R, Reuning-Scherer J, Gulanski B. The Alzheimer's disease assessment scale: patterns and predictors of baseline cognitive performance in multicenter Alzheimer's disease trials. Neurology 1997;48:1511-1517). A score of 15.2 on the MMSE corresponds to a value of approximately 36.5 on the ADAS-cog. In the case that the null hypothesis is false, the expected mean score of patients treated with interferon beta should be equal to 26.5 (given an SD=10). Similar to the previous study objective, the randomization of 17 patients to each group will permit the rejection of the null hypothesis with an alpha equal to 0.05 and power of 80%.

Considering a drop out rate of approximately 15%, the final estimate of sample size is of 20 patients per arm.

All serious adverse events (SAEs) reported while patients are on-study or within 30 days after discontinuing treatment are tabulated.

Laboratory tests at baseline and change from baseline are summarized by randomized treatment group. In addition, shift tables for laboratory tests based on a classification of values as low, normal or high with respect to the reference range are summarized and presented by randomized treatment group.

Assignment

The randomisation schedule is generated in the research pharmacy; the investigator and study personnel remain blinded to the group assignment of participants until the completion of data collection.

Outcome Measures

Outcome measures are assessed at baseline, week 12, week 28, and 52 (study completion).

Primary outcome measures included:

Alzheimer's Disease Assessment Scale (ADAS), cognitive subscale
Global Deterioration Scale
Clinical Global Impression of Change Scale
   Secondary outcome measures included:
MMSE
ADAS, non-cognitive subscale
Instrumental Activities of Daily Living (IADL)
Physical Self-Maintenance Scale (PSMS)
Caregiver-rated Global Impression of Change (cGIC)
Geriatric depression scale (GDS)
Patients who discontinued the study for disease progression into two treatment arms
   Evaluation of Adverse Events

The appearance of treatment-related adverse events is assessed at each visit. Withdrawal from the study is warranted upon any of the following:
5) Patient request
6) Investigator request
7) Evidence of severe systemic disease
8) Evidence of severe treatment-rated (IFN β-1a) adverse events

### REFERENCES

1. Moran, M. A., Mufson, E. J., and Gomez-Ramos, P. (1993) Acta Neuropathol. 85, 362-369.

2. Silver, A (1974) The Biology of Cholinesterases, North-Holland, Amsterdam.

3. Kasa, P., Rakonczay, Z, and Gulya, K (1997) Prog. Neurobiol. 52, 511-535.

4. Geula, C., Mesulam, M. M., Saroff, D. M., and Wu, C. K. (1998) J. Neuropathol. Exp. Neurol. 57, 63-75.

5. Massoulie, J., Pezzementi, L., Bon, S., Krejci, E., and Vallette, F. M. (1993) Prog. Neurobiol. 41, 31-91.

6. Mesulam, M. M., and Geula, C. (1990) Adv. Neurol. 51, 235-240.

7. Brimijoin, S.; Koenigsberger, C. (1999) Environ. Health Perspect., 107 Supl. 1, 59-64.

8. Bigbee, J. W.; Sharma, K. V.; Chan, E. L; Bogler, O. (2000) Brain Res., 861, 354-362.

9. Anderson, R. B.; Key, B. (1999) Int. J. Dev. Neurosci., 17, 787-793.

10. Johnson, G.; Moore, S. W. (2000) Int. J. Dev. Neurosci., 18, 781-790.

11. Sharma, K. V.; Bigbee, J. W. (1998) J. Neurosci. Res., 53, 454-464.

12. Mu{tilde under (n)}oz, F. J.; Aldunate, R.; Inestrosa, N. C. (1999) Neuroreport, 10, 3621-3625.

13. Johnson, G.; Moore, S.W. (1999) Bloerm Blophys. Res. Commun., 258, 75 762.

14. Inestrosa, N. C., Alvarez, A., Perez, C. A., Moreno, R. D., Vicente, M., Linker, C., Cassanueva, O. I., Soto, C., and Garrido, J. (1996) Neuron 16, 881-891.

15. Alvarez, A., Alarcon, R., Opazo. C., Campos, E. O., Munoz. F. J., Calderon, F. H., Dajas, F., Gentry, M. K., Doctor, B. P., De Mello, F. G., and Inestrosa, N. C. (1998) J. Neurosci. 18,3213-3223.

16. Munoz, F. J., and Inestosa, N. C. (1999) FEBS Lett. 450, 205-209.

17. Matthew G. Cottingham, Michael S. Hollinshead, and David J. T. Vaux, (2002) Biochemistry, 41 (46),13539 -13547.

18. Manuela Bartolini, Carlo Bertucci, Vanni Cavrini and Vincenza Andrisano, (2003) Biochemioal Pharmacology, Volume 65, Issue 3, 407-416.

19. Bartus, R. T. (2000) Exp. Neurol., 163, 495-529.

20. Melo J B et al., (2003) Neurosci Res, 45(1) :117-27.

21. Nordberg A, Svensson A L (1998) Cholinesterase Inhibitors in the treatment of Alzheimer's disease: a comparison of tolerability and pharmacology. Drug Safety, 19, 465480.

22. Davis K I. et al. (1999) JAMA; 281, 1401-1406.

23. Blesa R. et al, (2003) Dementia and Geriatric Cognitive Disorders, 15, 79-87.

24. Wiodek, S. T.; Antosiewicz, J.; McCamnon, J. A.; Stratsma, T. P.; Gilson, M. K.; Briggs, J. M.; Humblet, C.; Sussman, J. L. (1996) Biopolymers, 38, 109-117.

25. Bartolucci, C.; Perola, E.; Cellal, L.; Brufani, M.; Lamba, D. (1999) Biochem., 38, 5714-5719.

26. Szegletes, T.; Mallender, W. D.; Thomas, P. J.; Rosenberry, T. L. (1999) Biochem., 38, 122-133.

27. Harel, M.; Kleywegt, G. J.; Ravelli, R B.; Silman, I.; Sussman, J. L. (1995) Structure, 3, 1355-1366.

28. Harel, M.; Schalk, I.; Ehret-Sabatier, L.; Bouet, F.; Goeldner, M.; Hirth, C.; Axelsen, P. H.; Silman, I.; Sussman, J. L. (1993) Proc. Natl. Acad. Sci. USA, 90, 9031-9035.

29. Jonnala R R et al., (2003) Synapse, 47(4), 262-269.

30. Woodruff-Pak D S et al., (2002) CNS Drug Rev, 8(4), 405-26.

31. in t'Veld B A, Rultenberg A, Hofman A, Launer L J, van Duljn C M. et al. (2001) Nons-teroidal antiinflammatory drugs and the risk of Alzheimer's disease. N. Engl. J. Med. 345, 1515-21

32. Blasko I, Apochal A, Boeck G, Hartmann T, Grubeck-Loebenstein B, Ransmayr G. (2001) Ibuprofen decreases cytokine-induced amy-loid beta production in neuronal cells. Neu-robiol. Dis. 8, 1094-101.

33. Weggen S, Eriksen J L, Das P, Sagi S A, Wang R. et al. (2001) A subset of NSAIDs lower amyloldogenic Abeta42 independently of cyclooxygenase activity. Nature 414, 212-16.

34. Watterson D M, Halech J. and Van Eldik L J (2002) Discovery of new chemical classes of synthetic ligands that suppress neunoinflammatory responses. J Mol Neurosci 19, 89-93.

35. Waring S C, Rocca W A, Petersen R C, O'Brien P C, Tangalos E G, Kokmen E. (1999) Post-menopausal estrogen replacement therapy and risk of AD: a population-based study. Neurology 52, 965-70

36. Greenfield J P, Leung L W, Cal D, Kaasik K, Gross R S, et al. (2002) Estrogen lowers Alzheimer beta-amyloid generation by stim-ulating trans-Golgi network vesicle biogen-ests. J. Biol. Chem. 277(12), 128-36.

37. Fassbender K, Simons M, Bergmann C, Stroick M, Lutjohann D, et al. (2001) Simvastatin strongly reduces levels of Alzheimer'dis-ease beta-amyloid peptides Abeta 42 and Abeta 40 in vitro and in vivo. Proc. Natl. Acad. Sci. USA 98, 5856-61.

38. Michaelis M L, Dobrowsky R T, and Li G (2002) Neurofibrillary pathology and microtubul stability. J Mol Neursci. 19, 289-293.

39. Lau L F, Schachter J B, Seymour P A, and Sanner M A (2002) Protein phosphorylation as a therapeutic target in Alzheimer's disease. Curr Top Med Chem 2, 395-15.

40. Michaelis M L, Ranciat N, Chen Y. Bechtel M, Ragan R. Hepperle M. Liu Y, and Georg G (1998) Protection against β-amyloid toxicity in primary neurons by paclitaxel [Taxol], J Neurochem 70, 1623-1627.

41. Carson J A, Turner A J. (2002) Neurochem., 81, 1.

42. Familletti, P. C., Rubinstein, S., and Pestka, S. (1981) A Convenient and Rapid Cytopathic Effect Inhibition Assay for Interferon, In Methods In Enzymology, Vol. 78 (S. Pestka, ed.), Academic Press, New York, 387-394.

43. Pestka, S. (1986) “Interferon Standards and General Abbreviations, in Methods in Enzymology” (S. Pestka, ed.), Academic Press, New York 119, 14-23.

44. Derynk R. et al. (1980) Nature 285, 542-547.

45. Shepard H. M. et al. (1981) Nature, 294, 563 -565.

46. Mark D. F. et al. (1984) Proc. Natl. Acad. Sci. U.S.A., 81(18), 5662-6666.

47. Boutros T., Croze E and Young V. W. (1997) Journal of Neurochemistry. 69, 939-946.

1-25. (canceled)

26. A method of treating Alzheimer's disease, Creutzfeld-Jakob disease or Gerstmann-Sträussler-Scheinker disease comprising the administration of a composition comprising interferon-β (IFN-β) or derivatives thereof to an individual having Alzheimer's disease, Creutzfeld-Jakob disease or Gerstmann-Sträussler-Scheinker disease.

27. The method according to claim 26, wherein said composition is administered in combination with a composition comprising an agent selected from the group consisting of cholinesterase inhibitors, Aβ toxicity lowering agents, hormone replacement agents, lipid lowering agents, secretase modulating agents, Aβ aggregation inhibitors, neurofibrillar inhibitors and β-amyloid catabolism inhibitors.

28. The method according to claim 27, wherein said compositions are administered as a single composition.

29. The method according to claim 27, wherein said compositions are administered simultaneously, sequentially or separately as separate compositions.

30. The method according to claim 26, wherein said Alzheimer's disease is an early-onset Alzheimer's disease.

31. The method according to claim 27, wherein said cholinesterase inhibitor is an acetylcholinesterase inhibitor, a butyrylcholinesterase inhibitor or both an acetylcholinesterase inhibitor and a butyrylcholinesterase inhibitor or a derivative of an acetylcholinesterase inhibitor, a butyrylcholinesterase inhibitor or both an acetylcholinesterase inhibitor and a butyrylcholinesterase inhibitor.

32. The method according to claim 27, wherein said agent is donepezil, rivastigmine, galantamine, tacrine, amiridine, minaprine, huperzine, huprine, bis-tetrahydroaminoacridine (bis-THA), imidazoles, 1,2,4-thiadiazolidinone, benazepine, 4,4′-bipyridine, indenoquinolinylamine, decamethonium, edrophonium, physostigmine, metrifonate, propidium, fasciculins, organophosphates, carbamates, imino 1,2,3,4-tetrahydrocyclopent[b]indole carbamates, N-pyrimidine 4-acetylaniline, 7-aryloxycoumarin, propargylamino carbamates, vitamin E, NOS inhibitors, ACh precursors or cholinergic receptor agonists.

33. The method according to claim 32, wherein said Ach precursors are choline or pyrrolidinecholine.

34. The method according to claim 32, wherein said cholinergic receptor agonists are muscarinic, nicotinic or α7-cholinergic receptor agonists.

35. The method according to claim 27, wherein said Aβ toxicity lowering agents are ibuprofen, indomethacin, sulindac sulfide, death associated protein kinase (DAPK) inhibitors, cyclooxygenases (COX-1 and -2) inhibitors, antioxidants, NMDA modulators, or MAO inhibitors.

36. The method according to claim 27, wherein said hormone replacement agent is estrogen.

37. The method according to claim 35, wherein said lipid lowering agents are 3-hydroxy-3-methyglutaryl coenzyme A (HMG-CoA) reductase inhibitors, statins, lovastatin, pravastatin, atorvastatin, simvastatin, fluvastatin, cerivastatin, rosuvastatin, compactin, mevilonin, mevastatin, visastatin, velostatin, synvinolin, rivastatin, itavastatin, pitavastatin, methyl-β-cyclodextrin, 7-dehydrocholesterol reductases, acyl co-enzyme A:cholesterol acyltransferase (ACAT) inhibitors or P13K inhibitors.

38. The method according to claim 27, wherein said secretase modulating agents are inhibitors of β-secretase inhibitors, inhibitors of γ-secretase inhibitors, inhibitors of both β-secretase and γ-secretase or α-secretase promoting molecules.

39. The method according to claim 39, wherein said β-secretase inhibitors are BACE or BACE2 inhibitors.

40. The method according to claim 39, wherein said γ-secretase inhibitors are difluoroketone-based compounds, hydroxy substituted peptide urea, alanine-phenylglycine derivatives, caprolactams, benzodiazepines, hexanamides, fenchylamine sulfonamide, bicyclic sulfonamide, isocoumarin, diaryl acetylene, imidazopyridine, polyoxygenerated aromatic structures and said a-secretase promoting molecules are protein kinase C activators, glutamate, carbachol, muscarinic agonists, neurotrophic agents, or coper (II) containing compounds.

41. The method according to claim 27, wherein said Aβ aggregation inhibitors are peptidyl inhibitors, analogs of the amyloid binding dyes Congo red and thioflavin T, analogs of the anti-cancer agent doxorubicin, antibiotics, benzofurans, inhibitors of serum amyloid protein (SAP) or metal chelating agents by addition of Cu²⁺, Zn²⁺ or Fe³⁺.

42. The method according to claim 27, wherein said neurofibrillar inhibitors are GSK3β inhibitors, cdk5 inhibitors, calpain inhibitors, or paclitaxel and related agents.

43. The method according to claim 27, wherein said β-amyloid catabolism inhibitors are zinc metalloproteinases, endothelin-converting enzyme, insulin-degrading enzymes, plasmin, or neprilysin inhibitors.

44. The method according to claim 26, wherein said IFN-β further comprises at least one moiety attached to one or more functional groups which occur as one or more side chains on the amino acid residues.

45. The method according to claim 44, wherein said moiety is a polyethylene moiety.

46. The method according to claim 26, wherein said IFN-β is administered at a dosage of about 1 to 50 μg per person per day, or about 10 to 30 μg per person per day or about 10 to 20 μg per person per day.

47. The method according to claim 26, wherein said IFN-β is administered daily or every other day.

48. The method according to claim 26, wherein said IFN-β is administered twice or three times per week.

49. The method according to claim 46, wherein said IFN-β is administered at a dosage of less than 100 μg/m² or less than 50 μg/m² or less than 10 μg/m² or less than 1 μg/m².

50. The method according to claim 26, wherein said IFN-β is administered subcutaneously.

51. The method according to claim 26, wherein said IFN-β is administered intramuscularly.

52. The method according to claim 26, wherein said IFN-β is administered intravenously.

53. An article of manufacture consisting of two separate compositions manufactured in a packaging unit, one composition containing IFN-β and the other one containing an Alzheimer's disease treating agent selected from the group consisting of cholinesterase inhibitors, AP toxicity lowering agents, hormone replacement agents, lipid lowering agents, secretase modulating agents, Aβ aggregation inhibitors, neurofibrillar inhibitors and β-amyloid catabolism inhibitors.

54. A pharmaceutical composition comprising IFN-β in combination with an Alzheimer's disease treating agent selected from the group consisting of cholinesterase inhibitors, Aβ toxicity lowering agents, hormone replacement agents, lipid lowering agents, secretase modulating agents, Aβ aggregation inhibitors, neurofibrillar inhibitors and β-amyloid catabolism inhibitors and one or more pharmaceutically acceptable excipients.
